(19) United States
(12) Patent Application Publication
(10) Pub. No.: US 2026/0179235 A1
(43) Pub. Date: Jun. 25, 2026

(54) Tumor Motion Model Determination Method and Electronic Device

(71) Applicant: OUR UNITED CORPORATION, Shaanxi (CN)

(72) Inventors: Hao Yan, Shaanxi (CN); Dalin Liu, Shaanxi (CN); Zhongya Wang, Shaanxi (CN); Jiuliang Li, Shaanxi (CN)

(21) Appl. No.: 19/431,306

(22) Filed: Dec. 23, 2025

(30) Foreign Application Priority Data
Dec. 23, 2024 (CN) 202411909957.8

(51) Int. Cl. G06T 7/246; A61N 5/10; G06T 7/33; G06T 7/73; G16H 20/40; G16H 50/50

(52) U.S. Cl. CPC G06T 7/248; A61N 5/1049; G06T 7/337; G06T 7/74; G16H 20/40; G16H 50/50; G06T 2207/10081; G06T 2207/10088; G06T 2207/30096; G06T 2207/30241

(57) ABSTRACT

The present disclosure provides a tumor motion model determination method and an electronic device. The method includes acquiring a scanning image of a target object during a positioning stage, and a respiratory signal of the target object; and determining a tumor motion model based on a planning image of the target object, a position of a tumor of the target object in the scanning image, and the respiratory signal. Herein, the tumor motion model is used to characterize an orientation change situation of the tumor of the target object during a respiratory process of the target object.

### CROSS-REFERENCE TO RELATED APPLICATION

This application claims priority to Chinese Patent Application No. 202411909957.8, filed Dec. 23, 2024, the disclosure of which is hereby incorporated by reference in its entirety.

### BACKGROUND OF THE INVENTION

### Field of the Invention

The present disclosure relates to the technical field of medical treatment, and in particular, to the technical field of tumor tracking, and more specifically to a tumor motion model determination method and an electronic device.

### Description of Related Art

During radiation therapy, maintaining precise positioning for a tumor is one of the key technologies of radiation therapy. During the radiation therapy, respiratory movement of a patient may cause significant changes in a position of a tumor in the chest or abdomen (such as lung, liver, or pancreas). Therefore, precise positioning for a chest or abdominal tumor has become a highly challenging issue.

At present, a plurality of projection images may be harvested after a positioning stage is completed, and a tumor motion model is established based on the plurality of projection images, so as to determine a position of a tumor based on the tumor motion model during a treatment stage.

### SUMMARY OF THE INVENTION

In a first aspect, the present disclosure provides a tumor motion model determination method, and the method includes:
acquiring a scanning image of a target object during a positioning stage, and a respiratory signal of the target object; and determining a tumor motion model based on a planning image of the target object, a position of a tumor of the target object in the scanning image, and the respiratory signal.

Herein, the tumor motion model is used to characterize an orientation change situation of the tumor of the target object during a respiratory process of the target object.

In a second aspect, a tumor motion model determination apparatus is further provided in the present disclosure, and the apparatus includes:
an acquisition unit, configured to acquire a scanning image of a target object during a positioning stage, and a respiratory signal of the target object;
a determination unit, configured to determine a tumor motion model based on a planning image of the target object, a position of a tumor of the target object in the scanning image, and the respiratory signal. The tumor motion model is used to characterize an orientation change situation of the tumor of the target object during a respiratory process of the target object.

In a third aspect, an electronic device is further provided in the present disclosure, and the electronic device includes: a processor and a memory configured to store instructions executable for the processor; where the processor is configured to execute the instructions to implement the tumor motion model determination method in the above-mentioned first aspect.

### BRIEF DESCRIPTION OF THE DRAWINGS

The accompanying drawings are intended for a better understanding of the present scheme, and do not constitute the limitation of the present disclosure, where:

FIG. 1 is a schematic diagram of a scenario of a radiation therapy system provided in the embodiments of the present disclosure.

FIG. 2 is a flowchart of a tumor motion model determination method provided in the embodiments of the present disclosure.

FIG. 3 is a schematic diagram of a coordinate system for an imaging point provided in the embodiments of the present disclosure.

FIG. 4 is a flowchart of another tumor motion model determination method provided in the embodiments of the present disclosure.

FIG. 5 is a flowchart of yet another tumor motion model determination method provided in the embodiments of the present disclosure.

FIG. 6 is a schematic diagram of a real-time determination for a position of a tumor in a target object provided in the embodiments of the present disclosure.

FIG. 7 is a schematic block diagram of an electronic device provided in the embodiments of the present disclosure.

### DESCRIPTION OF THE INVENTION

The technical solutions in the embodiments of present disclosure will be described below clearly and completely in conjunction with the accompanying drawings. Obviously, the described embodiments are merely a part of embodiments of the present disclosure, but not all of embodiments. All other embodiments obtained based on the embodiments of the present disclosure by those of ordinary skill in the art without paying any creative effort shall be included in the protection scope of the present disclosure.

In the description of the present disclosure, the word “exemplary” is used to represent “being as an example, instance, or illustration.” Any embodiment in the present disclosure described as “exemplary” is not necessarily to be illustrated as preferred or advantageous over other embodiments in the present disclosure. The following description is presented to enable any skilled in the art to implement and use the present disclosure. In the following description, details are set forth for purposes of explanation. It should be understood that the ordinary technical personnel in the art may recognize that the present disclosure may be implemented without using these specific details. In other instances, well-known structures and processes are not described in detail to avoid obscuring the description of the present disclosure with unnecessary details. Thus, the present disclosure is not intended to be limited to the shown embodiments, but to be consistent with the widest scope of principles and features disclosed in the present disclosure.

It should be noted that since the method in the embodiments of the present disclosure is executed in an image computer device, and processing objects of various image computer devices exist in the form of data or information, such as time, which is essentially time information. It can be understood that in subsequent embodiments, if size, quantity, position, etc., are mentioned, they all exist in the form of corresponding data for processing by the computer device, and specific details will not be repeated herein.

During radiation therapy, maintaining precise positioning for a tumor is one of the key technologies of radiation therapy. During the radiation therapy, respiratory movement of a patient may cause significant changes in a position of a tumor in the chest or abdomen (such as lung, liver, or pancreas). Therefore, precise positioning for a chest or abdominal tumor has become a highly challenging issue.

At present, a plurality of projection images may be harvested after a positioning stage (also referred to as setup stage) is completed, and a tumor motion model is established based on the plurality of projection images, so as to determine a position of a tumor based on the tumor motion model during a treatment stage. But this manner is too complex and requires a lot of time.

Based on the above technical issues, a tumor motion model determination method is provided in the embodiments of the present disclosure. After acquiring the scanning image of the target object in the positioning stage and the respiratory signal of the target object, the tumor motion model may be determined based on the planning image of the target object, the position of the tumor of the target object in the scanning image, and the respiratory signal. Further, the tumor motion model is used to characterize an orientation change situation of the tumor of the target object during a respiratory process of the target object.

Through the above technical solutions, the tumor motion model of the target object may be directly obtained based on the scanning image acquired during the positioning stage, and the planning image and the respiratory signal of the target object without additional acquisition for multiple projection images to determine the tumor motion model after the positioning stage is completed. In this way, steps of determining the tumor motion model may be effectively simplified, time required for determining the tumor motion model may be effectively shortened, thus to save time.

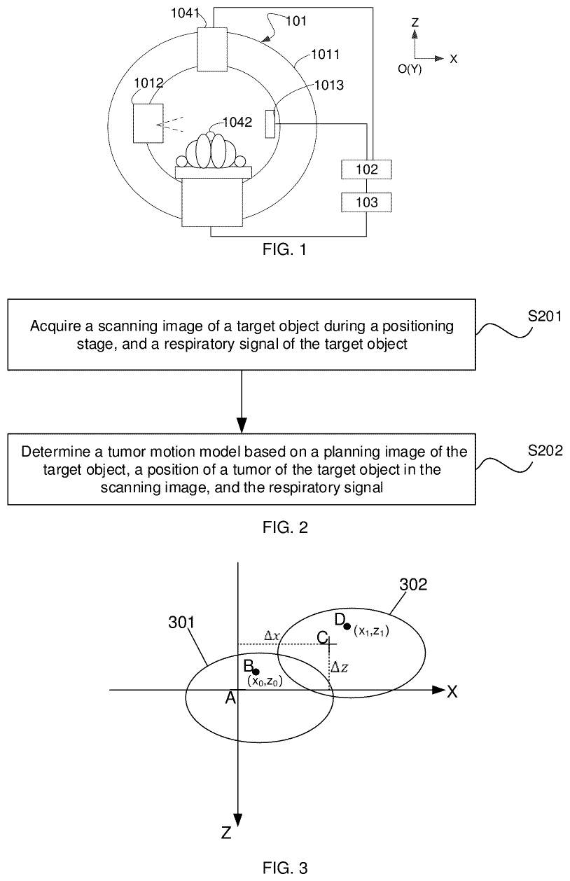

FIG. 1 is a schematic diagram of a radiation therapy system provided in the embodiments of the present disclosure, and the system may include an image guidance radiation therapy device 101, an image computer device 102, a control apparatus 103, and a respiration detection apparatus.

The image guidance radiation therapy device 101 may include a gantry 1011 and an image-guided apparatus mounted on the gantry 1011, where the image-guided apparatus includes a radiation source 1012 and a detector 1013. The radiation source 1012 is configured to emit a radiation beam, and the detector 1013 is configured to receive a radiation beam passing through the target object (i.e., patient) to generate a projection image of the target object.

In the embodiments of the present disclosure, the detector 1013 may be a flat-panel detector or a curved-surface detector. The shape of the detector 1013 may not be specifically limited in the embodiments of the present disclosure.

In the embodiments of the present disclosure, the image-guided apparatus may be at least one of: a cone beam computed tomography (CBCT) apparatus, a computed tomography (CT) apparatus, or a magnetic resonance (MR) apparatus. That is, the image-guided apparatus may be a CBCT apparatus, a CT apparatus, or an MR apparatus, or may include any two of a CBCT apparatus, a CT apparatus, and an MR apparatus. The image-guided apparatus may also include a CBCT apparatus, a CT apparatus, and an MR apparatus. The shape and form of the image-guided apparatus may not be specifically limited in the embodiments of the present disclosure.

In a case where the image-guided apparatus is a CBCT apparatus, the radiation source 1012 is an X-ray tube and the detector 1013 is a flat-panel detector.

In the embodiments of the present disclosure, there is no limitation on the number of radiation sources 1012 and the number of the detectors 1013. For example, the number of radiation sources 1012 may be one or multiple. Similarly, the number of detectors 1013 may be one or multiple. In a case where the number of radiation sources 1012 and the number of detectors 1013 are multiple, a plurality of two-dimensional images (i.e., projection images, also referred to as kilovolt (KV) images) for interior two-dimensional (2D) planes of the target object may be generated at a certain gantry angle (or time point).

In some embodiments, in a case where the number of radiation sources 1012 and the number of detectors 1013 are one, the radiation source and the detector may be located in the direction of the Z-axis shown in FIG. 1, so that the positions of the tumor of the target object in the X-axis direction and Y-axis direction may be obtained. Herein, the position of the tumor of the target object in the Y-axis direction represents a position of the tumor of the target object in a head-to-foot direction. The position of the tumor of the target object in the X-axis direction represents a position of the tumor of the target object in a left-to-right direction (i.e., on the left or right with respect to the target object).

In some embodiments, in a case where the number of radiation sources 1012 and the number of detectors 1013 are both two, one combination of a radiation source and a detector may be located in the Z-axis direction shown in FIG. 1 to obtain positions of the tumor of the target object in the X-axis direction and Y-axis direction, and another combination of a radiation source and a detector may be located in the X-axis direction shown in FIG. 1 to obtain positions of the tumor of the target object in the Z-axis direction and Y-axis direction. In this way, a three-dimensional spatial position of the tumor of the target object may be obtained based on the positions of the tumor of the target object in the X-axis direction and Y-axis direction and the positions of the tumor of the target object in the Z-axis direction and Y-axis direction. The position of the tumor of the target object in the Z-axis direction represents a position of the tumor of the target object in an front-to-behind direction (i.e., on the front or behind the target object, which is shown as up or down direction).

The gantry 1011 may be a ring gantry, a C-arm gantry, a drum gantry, a multi-layer bowl-shaped/cylindrical structure gantry, etc. The gantry 1011 may be a rotation gantry that is capable of moving around a rotation axis or an immovable fixed gantry. In a case where the gantry 1011 rotates, the radiation source 1012 and detector 1013 may rotate around the Y-axis by any angle, thus generating a two-dimensional image (i.e., projection image) of the target object in any 2D plane.

The respiration detection apparatus is detection to detect a respiratory signal of the target object.

In the embodiments of the present disclosure, the respiration detection apparatus may include an optical camera 1041 and at least one optical marker 1042 provided on the surface of the chest of the target object. For example, the optical camera 1041 may be an infrared camera, and correspondingly, the optical marker 1042 may be an infrared marker, or they may be other types of optical cameras and compatible optical markers. The shape and form of the respiration detection apparatus are not specifically limited in the embodiments of the present disclosure.

The image computer device 102 is respectively connected to the control apparatus 103, the detector 1013, and the respiration detection apparatus for communication. The control apparatus 103 is connected to a supporting apparatus for supporting the target object for communication. The control apparatus 103 is configured to control motion of the supporting apparatus based on an offset sent from the image computer device 102, thereby adjusting the position of the target object.

In some embodiments, the image computer device 102 is a computer device having a graphical user interface (GUI), and the computer device includes: one or more processors, a memory, and one or more application programs. For example, the image computer device 102 may include an image guidance system (IGS) application program. A processor of the image computer device executes the IGS application program to implement following operations: acquiring a scanning image of a target object during a positioning stage, and a respiratory signal of the target object; and determining a tumor motion model based on a planning image of the target object, a position of a tumor of the target object in the scanning image, and the respiratory signal. Herein, the tumor motion model is used to characterize an orientation change situation of the tumor of the target object during a respiratory process of the target object.

In the embodiments of the present disclosure, the image computer device 102 and the control apparatus 103 may be independent servers, or may be a server network or server cluster composed of servers. For example, the computer device described in the embodiments of the present disclosure includes, but not limited to: a computer, a network host, a single network server, a set of multiple network servers, or a cloud server composed of multiple servers. Herein, a cloud server is composed of a large number of computers or network servers based on cloud computing.

In the embodiments of the present disclosure, the image computer device 102 and the control apparatus 103 may be general-purpose computer devices or special-purpose computer devices. In a specific implementation, the computer device may be a desktop computer, a portable computer, a network server, a palmtop computer (personal digital assistant, PDA), a mobile phone, a tablet computer, a wireless terminal device, a communication device, an embedded device, etc., and a type of computer device is not limited in the embodiment.

The following will describe the tumor motion model determination method provided in the embodiments of the present disclosure in combination with FIG. 1 and taking the image computer device in FIG. 1 with one radiation source and one detector as an example. It should be noted that the tumor motion model determination method provided in the embodiments of the present disclosure may be performed during the positioning stage.

FIG. 2 is a flowchart of a tumor motion model determination method provided in the embodiments of the present disclosure. As shown in FIG. 2, the method includes the following S201-S202.

In S201, a scanning image of a target object during a positioning stage, and a respiratory signal of the target object are acquired.

The scanning image includes a plurality of projection images, and a projection image is a two-dimensional image.

Specifically, the image computer device may store a harvesting frequency and a harvesting duration. During the positioning stage before the radiation therapy y, the image computer device may acquire the harvesting frequency and harvesting duration, and send the harvesting frequency and harvesting duration to the image-guided apparatus and the respiration detection apparatus, respectively.

After the image-guided apparatus receives the harvesting frequency and harvesting duration, the image-guided apparatus may control a radiation source to emit radiation beams according to the harvesting frequency during the harvesting duration. A radiation beam emitted from the radiation source passes through the target object and reaches the detector, thus the detector may obtain a projection image of the target object during the positioning stage. The detector may send projection images of the target object during the positioning stage to the image computer device. By repeating the above steps, the image computer device may obtain the plurality of projection images of the target object during the positioning stage, namely the scanning image, such as a CBCT scanning image.

After the respiration detection apparatus receives the harvesting frequency and harvesting duration, the respiration detection apparatus may detect a respiratory state of the target object according to the harvesting frequency during the harvesting duration, so as to obtain the respiratory signal of the target object, and send the respiratory signal of the target object to the image computer device. In this way, the image computer device may obtain the respiratory signal of the target object.

The process of the respiration detection apparatus detecting the respiratory signal of the target object may refer to relevant technologies, which will not be repeated herein.

Herein, the harvesting frequency and the harvesting duration may be preset.

In the embodiments of the present disclosure, the harvesting frequency may be 9 Hertz (Hz) or may be 10 Hz, and there is no specific limitation on the value of the harvesting frequency in the embodiments of the present disclosure.

The harvesting duration may be set according to a duration of one respiratory cycle. For example, assuming that the duration of a complete respiratory cycle is 5 seconds(s), then the harvesting duration may be 10 s or 15 s, and there is no specific limitation on the value of the harvesting duration in the embodiments of the present disclosure.

In S202, a tumor motion model is determined based on a planning image of the target object, a position of a tumor of the target object in the scanning image, and the respiratory signal.

Herein, the planning image (such as a CT image) is an image obtained by performing tomographic imaging on the tumor of the target object upon formulating a treatment plan for the target object.

The tumor motion model is used to characterize an orientation change situation of the tumor of the target object during a respiratory process of the target object.

Specifically, the image computer device may acquire and store the planning image of target object. After obtaining the scanning image during the positioning stage and the respiratory signal of the target object, the image computer device may acquire a planning image of the target object stored in the image computer device itself, and determine the tumor motion model based on the planning image of the target object, the position of the tumor of the target object in the scanning image, and the respiratory signal.

Through the above technical solutions, the image computer device may obtain the tumor motion model of the target object directly based on the scanning image acquired during the positioning stage, and the planning image and the respiratory signal of the target object without additional acquisition for multiple projection images to determine the tumor motion model after the positioning stage is completed. In this way, steps of determining the tumor motion model may be effectively simplified, time required for determining the tumor motion model may be effectively shortened, thus to save time.

In an implementation, the image computer device may further determine a distance between a position of the tumor of the target object in the planning image and an imaging point before performing the above-mentioned S202. Correspondingly, the above-mentioned S202 may be replaced by: in response to that the distance between the position of the tumor of the target object in the planning image and the imaging point is shorter than or equal to a preset threshold, determining the tumor motion model based on the planning image, the position of the tumor of the target object in the scanning image, and the respiratory signal.

Herein, the imaging point is manually annotated by a medical worker in the planning image of the target object during a process of formulating the treatment plan.

In the embodiments of the present disclosure, the position of the tumor of the target object may be represented by a position of a centroid of the tumor, or may be represented by a position of a center of the tumor, or may be represented by a position of a center of gravity of the tumor, and the position of the tumor of the target object is not limited in the embodiments of the present disclosure.

In the embodiments of the present disclosure, the preset threshold (thresh) may be 9 millimeters (mm) or may be 5 mm, and there is no specific limitation on the value of the preset threshold in the embodiments of the present disclosure.

Specifically, the position of the tumor of the target object being represented by the position of the centroid of the tumor is taken as an example. As shown in FIG. 3, the ellipse 301 represents a planning image of the target object, point A is an imaging point annotated by a medical worker in the planning image of the target object, and point B is a centroid of the tumor of the target object annotated by the medical worker in the planning image. The image computer device may establish a coordinate system as shown in FIG. 3 with point A as a coordinate origin, a left-to-right direction of the target object as the X-axis, a front-to-behind direction (up-down direction in FIG. 1) of the target object as the Z-axis. The image computer device may determine the position of the centroid (i.e., a coordinate of point B) of the tumor of the target object in the planning image. The image computer device may determine the distance (i.e., a distance between point B and point A) between the position of the tumor of the target object in the planning image of the target object and the imaging point based on the following formula (1):

$$d = \sqrt{{x}_{0}^{2} + {z}_{0}^{2}} \qquad Formula ( 1 )$$

where d represents the distance between the position of the tumor of the target object in the planning image of the target object and the imaging point, and (x₀, z₀) represents a position of the centroid of the tumor of the target object in the planning image.

After determining the distance between the position of the tumor of the target object in the planning image and the imaging point through the above manner, the image computer device may compare the distance between the position of the tumor of the target object in the planning image and the imaging point with the preset threshold. In a case where the distance between the position of the tumor of the target object in the planning image and the imaging point is shorter than or equal to the preset threshold, the image computer device may determine the tumor motion model based on the planning image, the position of the tumor of the target object in scanning image, and respiratory signal. In a case where the distance between the position of the tumor of the target object in the planning image and the imaging point is greater than the preset threshold, it indicates that a magnification ratio of the tumor of the target object varies greatly at different angles, and the image computer device may not establish the tumor motion model.

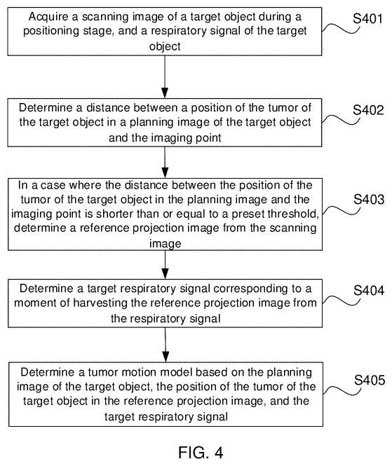

FIG. 4 is a flowchart of another tumor motion model determination method provided in the embodiments of the present disclosure. As shown in FIG. 4, the method includes the following S401-S405.

In S401, a scanning image of a target object during a positioning stage, and a respiratory signal of the target object are acquired.

A specific execution manner of S401 may refer to the description in S201 above, which will not be repeated herein.

In S402, a distance between a position of the tumor of the target object in a planning image of the target object and the imaging point is determined.

A specific execution manner of S402 may refer to the above-mentioned description, which will not be repeated herein.

In S403, in a case where the distance between the position of the tumor of the target object in the planning image and the imaging point is shorter than or equal to a preset threshold, a reference projection image is determined from the scanning image.

The reference projection image refers to an image containing the tumor of the target object among the plurality of projection images included in the scanning image.

A number of reference projection images is multiple. For example, the number of reference projection images may be 10, or may be 15, and the number of reference projection images is not limited in the embodiments of the present disclosure.

In an implementation, a process of the image computer device determining the reference projection image from the scanning image may specifically include: acquiring the reference projection image from projection images at a reference projection angle included in the scanning image according to a preset sampling interval.

The preset sampling interval may be 1. At this time, the image computer device may acquire a projection image as a reference projection image with an interval of one image from the projection images at the reference projection angle included in the scanning image. The preset sampling interval may be 2. At this time, the image computer device may acquire a projection image as a reference projection image with an interval of two images from the projection images at the reference projection angle included in the scanning image, which is not limited in the embodiments of the present disclosure.

The reference projection angle is determined according to at least one of a first projection angle, a second projection angle, and a third projection angle. Herein, the first projection angle refers to a projection angular range where a path of a radiation beam emitted from a radiation source to the tumor of the target object does not contain a preset tissue. The second projection angle refers to a projection angular range corresponding to a respiratory cycle during which a fitting degree between a motion trajectory of the tumor and a preset trajectory is highest. The third projection angle refers to an available angular range for half field imaging (the third projection angle may also be referred to as HF available angle).

The following will illustrate processes of the image computer device determining the first projection angle, second projection angle, and third projection angle.

### (1) Determine the First Projection Angle.

In an implementation, the planning image may contain contours of a plurality of tissues (such as liver, lungs, heart, spine, etc.) that are automatically or manually marked, and the plurality of tissues include the tumor of the target object. On this basis, the image computer device may determine an angle at which a path of the radiation beam emitted from the radiation source to the tumor of the target object in the planning image of the target object does not contain a preset tissue as the first projection angle by adopting a digital reconstruction projection algorithm after the image computer device acquires the planning image of the target object.

Herein, the preset tissue may include other tissues among the above-mentioned plurality of tissues excluding for the tumor of the target object.

Specifically, after the image computer device acquires the planning image of the target object stored by the image computer device itself, the image computer device may use a preset path simulation algorithm based on positions of the plurality of tissues in the planning image and the position of the radiation source, so as to simulate paths (also referred to as radiation paths) of the radiation beam emitted from the radiation source to the detector with passing through the tumor of the target object at respective projection angles. A target projection angle for at least one path that does not contain any other tissues except for the tumor of the target object is determined, that is, at the first projection angle, the radiation beam emitted from the radiation source may be free from interference from other objects (such as liver, lungs, heart, spine, etc.) in the planning image except for the tumor.

In the embodiments of the present disclosure, the path simulation algorithm may be a digitally reconstructed radiograph (DRR) algorithm, or may be a ray tracing algorithm, or a Monte Carlo algorithm, and the path simulation algorithm is not limited in the embodiments of the present disclosure.

### (2) Determine the Second Projection Angle.

In an implementation, the image computer device may determine a motion trajectory (referred to as a first motion trajectory below) of the tumor based on positions of the tumor of the target object in respective projection images included in the scanning image; determine a sub motion trajectory where the fitting degree is highest with the preset trajectory from the first motion trajectory; and finally take an angular range for harvesting projection images during the respiratory cycle corresponding to the sub motion trajectory as the second projection angle.

In the embodiments of the present disclosure, the preset trajectory may coincide with a curve of the Sine function or may coincide with a curve of the Cosine function, and there is no specific limitation on the preset trajectory in the embodiments of the present disclosure.

Specifically, taking the preset trajectory coinciding with the curve of the Sine function as an example, after the image computer device determines the positions of the tumor of the target object in the respective projection images included in the scanning image, the image computer device may generate the first motion trajectory of the tumor of the target object based on the positions of the tumor of the target object in the respective projection images and moments for harvesting the respective projection images. Afterwards, the image computer device may determine the sub motion trajectory with the highest fitting degree with the curve of the Sine function from the first motion trajectory. The image computer device may designate the respiratory cycle corresponding to the sub motion trajectory as the optimal respiratory cycle, and taking the angular range for harvesting the projection images during the optimal respiratory cycle as the second projection angle.

### (3) Determine the Third Projection Angle.

In an implementation, the image computer device may determine positions of the tumor projected on a plane where the detector is located at different angles; for any angle among the angles, in response to that a position of the tumor projected on the plane where the detector is located at that angle is within a region where the detector is located, determine the angle as the third projection angle.

Specifically, the position of the tumor of the target object being represented by the position of the centroid of the tumor is taken as an example. For any angle, the image computer device may determine the position of the tumor projected on the plane where the detector is located based on the angle, the position of the tumor of the target object in the scanning image, the distance between the radiation source and the detector, and the distance between the radiation source and the imaging point. In a case where the position of the tumor projected on the plane where the detector is located at the angle is within the region where the detector is located, the image computer device may determine this angle as the third projection angle.

Herein, the position of the tumor of the target object in the scanning image refers to a position of the tumor in a three-dimensional reconstructed image (e.g., a CBCT image) obtained by perform three-dimensional reconstruction on the scanning image.

The image computer device may determine the position of the tumor of the target object in the scanning image according to: acquiring a first offset, and determining the position of the tumor of the target object in the scanned image based on the first offset and the position of the tumor of the target object in the planning image.

Herein, the first offset is an offset obtained by performing registration on the scanning image and the planning image.

In an implementation, the image computer device may determine the first offset according to: performing three-dimensional reconstruction on the scanning image of the target object obtained during the positioning stage to obtain a three-dimensional reconstructed image. Registration is performed on the three-dimensional reconstructed image with the planning image of the target object to obtain the first offset.

The first offset may include a displacement offset (including displacement offsets on the X-axis, Y-axis, and Z-axis, respectively) and a rotation offset (including rotation offsets on the X-axis, Y-axis, and Z-axis, respectively). That is, the first offset may include: a displacement offset Δx and a rotation offset for the position of the tumor of the target object in the planning image with respect to the three-dimensional reconstructed image on the X-axis; a displacement offset Δz and a rotation offset for the position of the tumor of the target object in the planning image with respect to the three-dimensional reconstructed image on the Z-axis; a displacement offset Δy and a rotation offset for the position of the tumor of the target object in the planning image with respect to the three-dimensional reconstructed image on the y-axis.

It should be noted that the distance between the position of the tumor of the target object in the harvested scanning image of the target object and the imaging point upon harvesting the scanning image of the target object is consistent with the distance between the position of the tumor of the target object in the planning image and the imaging point annotated by the medical worker. Therefore, the first offset mentioned above is the same as an offset between the position of the imaging point upon the image computer device harvesting the scanning image of the target object during the positioning stage and the position of the imaging point annotated by the medical worker in the planning image.

Exemplarily, the first offset including the displacement offset Δx on the X-axis and the displacement offset Δz on the Z-axis is taken as an example. Continuing to refer to FIG. 3, the ellipse 302 represents a CBCT image, the point C is the position of the imaging point upon harvesting the scanning image of the target object during the positioning stage, and the point D is the centroid of the tumor of the target object in the CBCT image. The image computer device may determine the position x₁ of the tumor of the target object on the X-axis in the scanning image through formula (2), and determine the position z₁ of the tumor of the target object on the Z-axis in the scanning image through formula (3):

$${x}_{1} = {x}_{0} + Δ x \qquad Formula ( 2 )$$

$${z}_{1} = {z}_{0} + Δ z \qquad Formula ( 3 )$$

After determining the position of the tumor of the target object in the scanning image through the above manner, for any angle, the image computer device determines the position of the tumor projected on the plane where the detector is located at the angle through the following formula (4):

$$u = \frac{s i d * ( {x}_{1} cos ( θ ) + {z}_{1} sin ( θ ) )}{s a d - {x}_{1} cos ( θ ) + {z}_{1} sin ( θ )} \qquad Formula ( 4 )$$

where sid represents the distance between the radiation source and the detector, e represents the angle, sad represents the distance between the radiation source and the imaging point, and (x₁, z₁) represents the position of the centroid of the tumor of the target object in the scanning image.

By repeating the above steps, the image computer device may obtain the positions of the tumor projected on the plane where the detector is located at various angles. For each angle, the image computer device may compare the position of the tumor projected on the plane where the detector is located at that angle with the region where the detector is located. In a case where the position of the tumor projected on the plane where the detector is located at that angle is within the region where the detector is located, the image computer device may determine that angle as the third projection angle.

After determining the first projection angle, second projection angle, and third projection angle through the above manners, the image computer device may take an intersection of at least two projection angles from the first projection angle, second projection angle, and third projection angle as the reference projection angle.

In an implementation, in a case where an imaging mode is a half field imaging mode, the image computer device may take the intersection of the first projection angle and the third projection angle as the reference projection angle.

In S404, a target respiratory signal corresponding to a moment of harvesting the reference projection image is determined from the respiratory signal.

Specifically, after determining the reference projection image, the image computer device may determine the moment (hereinafter referred to as moment A) of harvesting the reference projection image. Afterwards, the image computer device may take a respiratory signal corresponding to the moment A as the target respiratory signal.

In S405, a tumor motion model is determined based on the planning image of the target object, the position of the tumor of the target object in the reference projection image, and the target respiratory signal.

In an implementation, before performing S405, the image computer device may further acquire the first offset, and then determine the tumor motion model based on the first offset with referring to the following manner a, manner b, or manner c.

### Manner a:

An adjusted planning image is acquired from adjustment based on a first offset, and for each of the reference projection images, the image computer device may perform digital reconstruction on the adjusted planning image at a projection angle for harvesting the reference projection image to obtain a first DRR image, and perform registration on the reference projection image and the first DRR image to obtain a second offset corresponding to the reference projection image. Finally, the tumor motion model is determined based on second offsets corresponding to respective reference projection images and the target respiratory signal.

Herein, a second offset refers to a relative offset between a position of the tumor in the reference projection image and a position of the tumor in the first DRR image.

Specifically, taking a reference projection image as a KV image B as an example, assuming that a projection angle for harvesting the KV image B is angle P, the first DRR image obtained by performing digital reconstruction on the adjusted planning image of the target object at the angle P is DRR image b.

After the image computer device obtains the DRR image b, the image computer device may perform registration on the DRR image b with the KV image B to obtain the position of the tumor of the target object in the KV image B. The image computer device may use the position of the tumor of the target object in the KV image B, the position of the tumor of the target object in the DRR image b, and a magnification ratio for the tumor upon harvesting the KV image B to obtain the second offset corresponding to the KV image B.

For example, assuming that the position of the tumor of the target object in the KV image B is p_{cbct}(P), and the position of the tumor of the target object in the DRR image b is p_{drr}(P), the magnification ratio for the tumor upon harvesting the KV image B is M(P)_{cbct}, the image computer device may obtain the second offset D(P) corresponding to the KV image B by adopting the following formula (5):

$$D ( P ) = \frac{{p}_{cbct} ( P ) - {p}_{drr} ( P )}{{M ( P )}_{cbct}} \qquad Formula ( 5 )$$

Herein, the magnification ratio M(P)_{cbct} for the tumor upon harvesting the KV image B may be determined through the following formula (6):

$${M ( θ )}_{c b c t} = \frac{s i d}{s a d - {x}_{1} cos ( P ) + {y}_{1} sin ( P )} \qquad Formula ( 6 )$$

By repeating the above steps, the image computer device may obtain the second offsets corresponding to the respective reference projection images.

The image computer device may generate a second motion trajectory of the tumor of the target object based on the second offsets corresponding to the respective reference projection images and moments of harvesting the respective reference projection images. The image computer device obtain the tumor motion model of the target object based on the second motion trajectory of the tumor and the target respiratory signal by adopting a motion model generation method.

In the embodiments of the present disclosure, the motion model generation method may be a polynomial fitting method, a linear regression method, or a neural network method (such as a multi-layer perceptron), which is not limited in the embodiments of the present disclosure.

Obtaining the tumor motion model of the target object based on the second motion trajectory of the tumor of the target object and the target respiratory signal may refer to relevant technologies, which will not be repeated herein.

### Manner b:

For each of the reference projection images, the image computer device may perform digital reconstruction on the planning image of the target object at a projection angle for harvesting the reference projection image to obtain a DRR image (which is referred to as second DRR image below), and perform registration on the reference projection image and the second DRR image to obtain a third offset corresponding to the reference projection image. The image computer device may adjust third offsets corresponding to respective reference projection images by adopting the first offset; and determine the tumor motion model based on the adjusted third offsets corresponding to the respective reference projection images and the target respiratory signal.

Herein, a third offset refers to a relative offset between the position of the tumor in the reference projection image and a position of the tumor in the second DRR image.

Specifically, taking a reference projection image as a KV image A as an example, assuming that a projection angle for harvesting the KV image A is angle θ, the second DRR image obtained by performing digital reconstruction on the planning image of the target object at the angle θ is DRR image a.

After the image computer device obtains the DRR image a, the image computer device may perform registration on the DRR image a with the KV image A to obtain the position of the tumor of the target object in the KV image A. The image computer device may determine a true position of the tumor in the body of the target object in KV image A with the position of the tumor of the target object in KV image A and the magnification ratio for the tumor upon harvesting the KV image A. The image computer device may further determine the true position of the tumor in the body of the target object in the DRR image a with the position of the tumor of the target object in the DRR image a and the magnification ratio for the tumor upon performing scanning for the planning image at the angle θ.

Afterwards, the image computer device may obtain the third offset corresponding to the KV image A based on the true position of the tumor in the body of the target object in the KV image A and the true position of the tumor in the body of the target object in the DRR image a. The image computer device may superimpose an offset of the first offset in a head-to-foot direction (i.e., the Y-axis) and the third offset corresponding to the KV image A to obtain an adjusted third offset corresponding to the KV image A.

For example, assuming that the position of the tumor of the target object in the KV image A is p_{cbct}(θ), and the position of the tumor of the target object in the DRR image a is p_{drr}(θ) the magnification ratio for the tumor upon harvesting the KV image A is M(θ)_{cbct}) and the magnification ratio for the tumor upon performing scanning for the planning image at the angle θ is M(θ)dᵣᵣ, the image computer device may obtain the third offset corresponding to KV image A being D(θ) by using the following formula (7):

$$D ( θ ) = \frac{{p}_{c b c t} ( θ )}{{M ( θ )}_{c b c t}} - \frac{{p}_{drr} ( θ )}{{M ( θ )}_{drr}} \qquad Formula ( 7 )$$

Herein, the magnification ratio for the tumor upon harvesting the KV image A being M(θ)_{cbct} may be determined through the above-mentioned formula (6), and the magnification ratio for the tumor upon performing scanning for the planning image at the angle θ being M(θ)dᵣᵣ may be determined through formula (8) below:

$${M ( θ )}_{d r r} = \frac{s i d}{s a d - {x}_{0} cos ( θ ) + {y}_{0} sin ( θ )} \qquad Formula ( 8 )$$

After the image computer device obtains the third offset corresponding to the KV image A through the above formulas, the image computer device may adopt formula (9) to superimpose an offset Δy in the head-to-foot direction (i.e., the Y-axis) with the third offset corresponding to the KV image A, to obtain the adjusted third offset D(θ)′ corresponding to the KV image A:

$${D ( θ )}^{′} = D ( θ ) + Δy \qquad Formula ( 9 )$$

By repeating the above steps, the image computer device may obtain the adjusted third offsets corresponding to the respective reference projection images. The image computer device may determine the tumor motion model based on the adjusted third offsets corresponding to the respective reference projection images and the target respiratory signal. Specific details may refer to the description in manner a, which will not be repeated herein.

### Manner c:

The positions of the tumor of the target object in the respective reference projection images are adjusted by adopting the first offset, and the tumor motion model is determined based on the adjusted positions of the tumor of the target object in the respective reference projection images and the target respiratory signal.

Specifically, for each reference projection image, the image computer device may perform digital reconstruction on the planning image of the target object at that projection angle corresponding to the reference projection image, to obtain the second DRR image corresponding to the reference projection image. The image computer device may perform registration on the second DRR image corresponding to the reference projection image with the reference projection image to obtain the position of the tumor of the target object in the reference projection image. The image computer device may adopt the first offset to adjust the position of the tumor of the target object in the reference projection image. By repeating the above steps, the image computer device may obtain respective adjusted reference projection images.

The image computer device may determine the tumor motion model based on the adjusted positions of the tumor of the target object in the respective reference projection images and the target respiratory signal. Specific details may refer to the description in manner a, which will not be repeated herein.

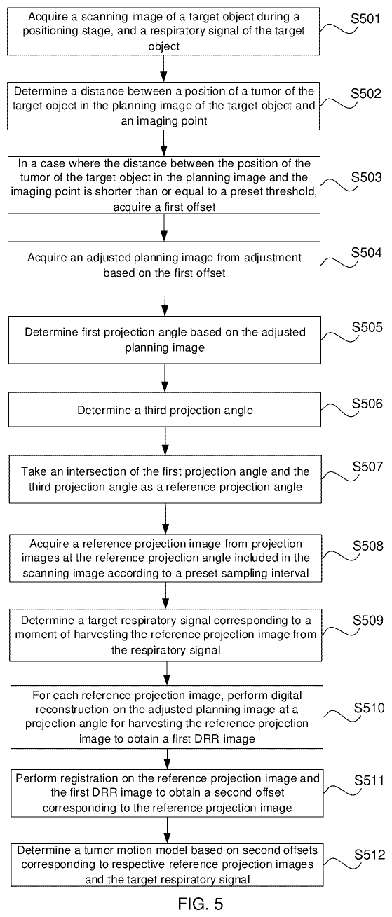

Taking the half field imaging mode as an example, the following introduces the method for tumor motion model determination method provided in the embodiments of the present disclosure. FIG. 5 is a flowchart of another tumor motion model determination method provided in the embodiments of the present disclosure. As shown in FIG. 2, the method includes the following S501-S512.

In S501, a scanning image of a target object during a positioning stage, and a respiratory signal of the target object are acquired.

In S502, a distance between a position of a tumor of the target object in the planning image of the target object and an imaging point is determined.

In S503, in a case where the distance between the position of the tumor of the target object in the planning image and the imaging point is shorter than or equal to a preset threshold, a first offset is acquired.

In S504, an adjusted planning image is acquired from adjustment based on the first offset.

In S505, a first projection angle is determined based on the adjusted planning image.

In S506, a third projection angle is determined.

In S507, an intersection of the first projection angle and the third projection angle is taken as a reference projection angle.

In S508, a reference projection image is acquired from projection images at the reference projection angle included in the scanning image according to a preset sampling interval.

In S509, a target respiratory signal corresponding to a moment of harvesting the reference projection image is determined from the respiratory signal.

In S510, for each reference projection image, digital reconstruction is performed on the adjusted planning image at a projection angle for harvesting the reference projection image to obtain a first DRR image.

In S511, registration is performed on the reference projection image and the first DRR image to obtain a second offset corresponding to the reference projection image.

In S512, a tumor motion model is determined based on second offsets corresponding to respective reference projection images and the target respiratory signal.

In some embodiments, the above-mentioned S504-S505 may be replaced with step a: the first projection angle is determined based on the planning image. On this basis, the above-mentioned S510-S512 may be replaced with the following steps b-d.

In step b, for each reference projection image, digital reconstruction is performed on the planning image at a projection angle for harvesting the reference projection image to obtain a second DRR image.

In step c, registration is performed on the reference projection image and the second DRR image to obtain a third offset corresponding to the reference projection image.

In step d, the tumor motion model is determined based on third offsets corresponding to respective reference projection images and the target respiratory signal.

In an implementation, after determining the tumor motion model through the above manner, the image computer device may determine the position of the tumor of the target object in real time based on the tumor motion model.

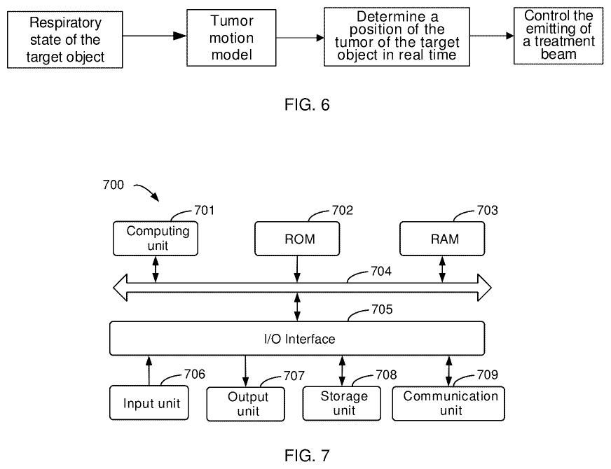

Specifically, as shown in FIG. 6, after obtaining the tumor motion model, in the real-time monitoring stage during the radiation therapy, the image computer device may obtain the respiratory state of the target object in real time through the respiration detection apparatus, and determine the offset of the position of the tumor of the target object with respect to the position of the tumor in the DRR image (e.g., the second DRR image or the first DRR image) in real time based on the respiratory state of the target object and the tumor motion model. The image computer device may superimpose offsets between the position of the tumor in the DRR image and the real-time determined positions of the tumor of the target object to obtain the position of the tumor of the target object, achieving prediction of tumor position.

Afterwards, the image computer device may control the emitting of a treatment beam through the control apparatus based on the determined position of the tumor of the target object to make it reach the tumor of the target object through a multi leaf collimator (MLC), achieving treatment for the tumor of the target object.

During the radiation therapy, if a motion center of the tumor of the target object is not aligned with a center of a target region, there may be a phenomenon where the tumor moves beyond the target region, resulting in the inability to treat the tumor that exceed the target region. In order to avoid the above phenomenon, in an implementation, in a case where the motion center of the tumor of the target object is inconsistent with the center of the target region, the image computer device may determine an adjustment parameter for adjusting the position of the target object during the positioning stage before the radiation therapy, and determine a tumor motion model corresponding to the adjusted position of the target object based on the adjustment parameter.

Herein, the target region is a region calibrated by a medical worker based on the position of the tumor of the target object in the planning image.

Specifically, the image computer device may first determine whether the motion center of the tumor of the target object is consistent with the center of the target region by referring to the following manner: a maximum value and a minimum value of the second motion trajectory of the tumor of the target object are acquired; in a case where an absolute value of the maximum value of the second motion trajectory is the same as an absolute value of the minimum value of the second motion trajectory, the image computer device may determine that the motion center of the tumor of the target object is consistent with the center of the target region. In a case where the absolute value of the maximum value of the second motion trajectory is different from the absolute value of the minimum value of the second motion trajectory, the image computer device may determine that the motion center of the tumor of the target object is not consistent with the center of the target region.

In a case where the motion center of the tumor of the target object is not consistent with the center of the target region, the image computer device may determine the adjustment parameter according to the maximum value and the minimum value of the second motion trajectory of the tumor of the target object by adopting the following formula (10), and send the adjustment parameter to the control apparatus. The control apparatus may control the supporting apparatus to move based on the adjustment parameter, so as to adjust the position of the target object:

$$deltaCouch = \frac{{V}_{max} + {V}_{min}}{2} \qquad Formula ( 10 )$$

where deltaCouch represents the adjustment parameter, Vₘₐₓ represents the maximum value of the second motion trajectory, Vₘᵢₙ represents the minimum value of the second motion trajectory.

Correspondingly, the above image computer device may determine the tumor motion model corresponding to the adjusted position of the target object by referring the following two manners.

Manner 1: the image computer device adjusts the second motion trajectory of the tumor of the target object based on the adjustment parameter, and determines the tumor motion model corresponding to the adjusted position of the target object based on the adjusted second motion trajectory and the target respiratory signal of the target object.

Specifically, assuming that the second motion trajectory of the tumor of the target object is trajectory K, and the adjusted second motion trajectory is trajectory K′, the image computer device may obtain the trajectory K′ by adopting the following formula (11):

$${K}^{′} = K - deltaCouch \qquad Formula ( 11 )$$

After obtaining the trajectory K′, the image computer device may obtain the tumor motion model corresponding to the adjusted position of the target object based on the trajectory K′ and the target respiratory signal of the target object.

Method 2: the image computer device adjusts a zero-order item (or a constant item) in a polynomial of the determined tumor motion model above of the target object based on the adjustment parameter, and obtains the tumor motion model corresponding to the adjusted position of the adjusted target object.

Specifically, assuming that the zero-order item in the tumor motion model is G, and the adjusted zero-order item is G′, the image computer device may obtain the adjusted zero-order item G′ by adopting the following formula (12):

$${G}^{′} = G - deltaCouch \qquad Formula ( 12 )$$

After obtaining the zero-order item G′, the image computer device may replace the zero-order item in the polynomial of the tumor motion model of the target object based on the zero-order item G′, to obtain the tumor motion model corresponding to the adjusted position of the target object.

FIG. 7 shows a schematic block diagram of an example of an electronic device 700 that is capable of being configured to implement the embodiments of the present disclosure. An electronic device is intended to represent various forms of digital computers, such as a laptop, a desktop computer, a workstation, a personal digital assistant, a server, a blade server, a mainframe computer, and other appropriate computers. The electronic device may also represent various forms of mobile apparatuses, such as a personal digital assistant, a cellular phone, a smart phone, a wearable device, and other similar computing apparatuses. The components shown herein, their connections and relationships, and their functions, are intended only as examples, and are not meant to limit implementations of the present disclosure described and/or claimed herein. In some embodiments, the electronic device may be the image computer device shown in above-mentioned FIG. 1.

As shown in FIG. 7, the electronic device 700 includes a computing unit 701, which may perform various appropriate actions and processes according to computer programs stored in a read-only memory (ROM) 702 or computer programs loaded from a storage unit 708 to a random access memory (RAM) 703. In RAM 703, various programs and data required for operations of the electronic device 700 may also be stored. The computing unit 701, ROM 702, and RAM 703 are connected to each other through the bus 704. An input/output (I/O) interface 705 is also connected to the bus 704.

Multiple components in the electronic device 700 are connected to the I/O interface 705, which include: an input unit 706 (such as a keyboard, a mouse, etc.); an output unit 707 (such as various types of displays, speakers, etc.); a storage unit 708 (such as a disk, an optical disk, etc.); and a communication unit 709 (such as a network card, a modem, a wireless communication transceiver, etc.). The communication unit 709 allows the electronic device 700 to exchange information/data with other devices through computer networks such as the Internet and/or various telecommunications networks.

The computing unit 701 may be a various general and/or special processing components having processing and computing capabilities. Some examples of the computing unit 701 include, but are not limited to, a central processing unit, a graphics processing unit (GPU), various dedicated artificial intelligence (AI) computing chips, various computing units that run machine learning model algorithms, a digital signal processor, and any appropriate processors, controllers, microcontrollers, etc. The computing unit 701 performs the various methods and processes as described above, such as the tumor motion model determination method. For example, in one embodiment, the tumor motion model determination method may be implemented as computer software programs, which are tangibly included in a machine-readable medium, such as the storage unit 708. In one embodiment, a portion or all of the computer programs may be loaded and/or installed on the electronic device 700 via the ROM 702 and/or the communication unit 709. In a case where the computer programs are loaded into the RAM 703 and executed by the computing unit 701, one or more steps of the tumor motion model determination method described above may be performed. Alternatively, in other embodiments, the computing unit 701 may be configured to perform the tumor motion model determination method in any other appropriate manners (e.g., by means of firmware).

Various implementations of the systems and techniques described above in the present document may be realized in a digital electronic circuit system, an integrated circuit system, a field programmable gate array, an application specific integrated circuit, application specific standard parts (ASSP), a system on chip (SOC), a complex programmable logic device (CPLD), a computer hardware, firmware, software, and/or combinations thereof. These various implementations may include: being implemented in one or more computer programs, where the one or more computer programs may be executed and/or interpreted on a programmable system including at least one programmable processor, and a programmable processor may be a special purpose or general purpose programmable processor that can receive data and instructions from a storage system, at least one input device, and at least one output device, and transmit data and instructions to the storage system, the at least one input device, and the at least one output device.

Program codes for implementing the methods of the present disclosure may be written in any combination of one or more programming languages. These program codes may be provided to a processor or a controller of a general-purpose computer, a special-purpose computer or other programmable data processing devices, so that when the program codes are executed by the processor or controller, the functions/operations specified in the flowcharts and/or block diagrams are implemented. The program codes may be executed entirely on the machine, executed partly on the machine, may be as a stand-alone software package, and partly executed on the machine and partly executed on a remote machine, or entirely executed on the remote machine or server.

In the context of the present disclosure, a machine-readable medium may be a tangible medium, which may contain or store programs for use by an instruction execution system, apparatus, or device, or for use in connection with the instruction execution system, apparatus, or device. The machine-readable medium may be a machine-readable signal medium or a machine-readable storage medium. The machine-readable medium may include, but is not limited to, an electronic, magnetic, optical, electromagnetic, infrared, or semiconductor system, apparatus, or device, or any suitable combination thereof. More specific examples of the machine-readable storage medium may include electrical connections based on one or more wires, a portable computer disk, a hard disk, a random access memory, a read-only memory, an erasable programmable read-only memory, optical fibers, a portable compact disk read-only memory, an optical storage device, a magnetic storage device, or any suitable combination thereof.

To provide interaction with a user, systems and techniques described herein may be implemented on a computer, and the computer has: a display apparatus (such as a cathode ray tube (CRT) or a liquid crystal display (LCD) monitor) for displaying information to the user; and a keyboard and a pointing apparatus (such as a mouse or trackball), and the user may provide input to the computer through the keyboard and the pointing apparatus. Other types of apparatuses may also be configured to provide interaction with the user; for example, a feedback provided to the user may be any form of sensory feedback (e.g., a visual feedback, an auditory feedback, or a tactile feedback); and input from the user may be received in any form (including an acoustic input, a voice input, or a tactile input).

The systems and techniques described herein may be implemented in a computing system that includes back-end components (e.g., as a data server), or a computing system that includes middleware components (e.g., an application server), or a computing system that includes front-end components (e.g., a user computer with a graphical user interface or a web browser, and a user may interact with implementations of the systems and techniques described herein through the web browser), or a computing system that includes any combination of such back-end components, middleware components, or front-end components. The components of the system may be interconnected by any form or media of digital data communication (e.g., a communication network). Examples of the communication network include a local area network (LAN), a wide area network (WAN), and the Internet.

A computer system may include a client and a server. The client and server are generally remote from each other and typically interact with each other through the communication network. A relationship of the client and server is arisen by computer programs running on the corresponding computers and having a client-server relationship to each other. The server may be a cloud server, or also may be a server of a distributed system, or a server combined with the blockchain.

It should be understood that various forms of the processes shown above may be used with steps being reordered, added or deleted. For example, the various steps described in the present disclosure may be executed in parallel, or may also be executed sequentially, or may be executed in different orders, as long as the expected results of the technical solutions in the present disclosure can be achieved, and the orders for executing the steps are not limited herein.

The specific implementations mentioned above do not limit the scope of the present disclosure. It should be understood by those skilled in the art that various modifications, combinations, sub-combinations and substitutions may be made according to design requirements and other factors. Any modifications, equivalent substitutions, and improvements made within the principles of the present disclosure shall be included within the scope of protection of the present disclosure.

What is claimed is:

1. A tumor motion model determination method, wherein the method comprises:
acquiring a scanning image of a target object during a positioning stage, and a respiratory signal of the target object; and
determining a tumor motion model based on a planning image of the target object, a position of a tumor of the target object in the scanning image, and the respiratory signal; wherein the tumor motion model is used to characterize an orientation change situation of the tumor of the target object during a respiratory process of the target object.

2. The method according to claim 1, wherein the method further comprises:
in response to that a distance between a position of the tumor of the target object in the planning image and an imaging point is shorter than or equal to a preset threshold, determining the tumor motion model based on the planning image, the position of the tumor of the target object in the scanning image, and the respiratory signal.

3. The method according to claim 1, wherein the scanning image comprises a plurality of projection images;
before determining the tumor motion model based on the planning image of the target object, the position of the tumor of the target object in the scanning image, and the respiratory signal, the method further comprises:
determining a reference projection image from the scanning image; wherein the reference projection image refers to an image containing the tumor of the target object among the plurality of projection images;
determining a target respiratory signal corresponding to a moment of harvesting the reference projection image from the respiratory signal; and
determining the tumor motion model based on the planning image of the target object, the position of the tumor of the target object in the scanning image, and the respiratory signal comprises:
determining the tumor motion model based on the planning image, a position of the tumor of the target object in the reference projection image, and the target respiratory signal.

4. The method according to claim 3, wherein determining the reference projection image from the scanning image comprises:
acquiring the reference projection image from projection images at a reference projection angle included in the scanning image according to a preset sampling interval.

5. The method according to claim 4, wherein the reference projection angle is determined according to at least one of a first projection angle, a second projection angle, and a third projection angle;
wherein the first projection angle refers to a projection angular range where a path of a radiation beam emitted from a radiation source to the tumor of the target object does not contain a preset tissue;
the second projection angle refers to a projection angular range corresponding to a respiratory cycle during which a fitting degree between a motion trajectory of the tumor and a preset trajectory is highest;
the third projection angle refers to an available angular range for half field imaging.

6. The method according to claim 5, wherein in a half field imaging mode, the reference projection angle is an intersection of the first projection angle and the third projection angle.

7. The method according to claim 5, wherein determining the second projection comprises:
determining the motion trajectory of the tumor based on positions of the tumor of the target object in respective projection images comprised in the scanning image;
determining a sub motion trajectory where the fitting degree is highest with the preset trajectory from the motion trajectory; and
taking an angular range for harvesting projection images during the respiratory cycle corresponding to the sub motion trajectory as the second projection angle.

8. The method according to claim 5, wherein determining the third projection comprises:
determining positions of the tumor projected on a plane where a detector is located at different angles;
for any angle among the angles, in response to that a position of the tumor projected on the plane where the detector is located at the angle is within a region where the detector is located, determining the angle as the third projection angle.

9. The method according to claim 8, wherein for any angle among the angles, determining the position of the tumor projected on the plane where the detector is located comprises:
determining the position of the tumor projected on the plane where the detector is located based on the angle, the position of the tumor of the target object in the scanning image, a distance between the radiation source and the detector, and a distance between the radiation source and an imaging point.

10. The method according to claim 5, wherein determining the first projection comprises:
determining the angle at which a path of the radiation beam emitted from the radiation source to the tumor of the target object in the planning image does not contain the preset tissue as the first projection angle by adopting a digital reconstruction projection algorithm.

11. The method according to claim 10, wherein before determining the angle at which the path of the radiation beam emitted from the radiation source to the tumor of the target object in the planning image does not contain the preset tissue as the first projection angle, the method further comprises:
acquiring a first offset; wherein the first offset is an offset obtained based on registration for the scanning image and the planning image;
adjusting the planning image based on the first offset; and
determining the angle at which the path of the radiation beam emitted from the radiation source to the tumor of the target object in the planning image does not contain the preset tissue as the first projection angle comprises:
determining an angle at which a path of the radiation beam emitted from the radiation source to the tumor of the target object in the adjusted planning image does not contain the preset tissue as the first projection angle.

12. The method according to claim 3, wherein a number of the reference projection images is multiple;
determining the tumor motion model based on the planning image of the target object, the position of the tumor of the target object in the scanning image, and the respiratory signal comprises:
acquiring an adjusted planning image from adjustment based on a first offset; wherein the first offset is an offset obtained based on registration for the scanning image and the planning image;
for each of the reference projection images, performing digital reconstruction on the adjusted planning image at a projection angle for harvesting the reference projection image to obtain a first digitally reconstructed radiograph (DRR) image, and performing registration on the reference projection image and the first DRR image to obtain a second offset corresponding to the reference projection image;
determining the tumor motion model based on second offsets corresponding to respective reference projection images and the target respiratory signal.

13. The method according to claim 3, wherein a number of the reference projection images is multiple;
determining the tumor motion model based on the planning image of the target object, the position of the tumor of the target object in the scanning image, and the respiratory signal comprises:
for each of the reference projection images, performing digital reconstruction on the planning image at a projection angle for harvesting the reference projection image to obtain a second digitally reconstructed radiograph (DRR) image, and performing registration on the reference projection image and the second DRR image to obtain a third offset corresponding to the reference projection image;
acquiring a first offset; wherein the first offset is an offset obtained based on registration for the scanning image and the planning image;
adjusting third offsets corresponding to respective reference projection images by adopting the first offset;
determining the tumor motion model based on the adjusted third offsets corresponding to the respective reference projection images and the target respiratory signal.

14. The method according to claim 3, wherein a number of the reference projection images is multiple;
determining the tumor motion model based on the planning image of the target object, the position of the tumor of the target object in the scanning image, and the respiratory signal comprises:
acquiring a first offset, wherein the first offset is an offset obtained based on registration for the scanning image and the planning image;
adjusting positions of the tumor of the target object in respective reference projection images;
determining the tumor motion model based on the adjusted positions of the tumor of the target object in the respective reference projection images, and the target respiratory signal.

15. An electronic device, wherein the electronic device comprises:
a processor; and
a memory configured to store instructions executable for the processor;
wherein the processor is configured to execute the instructions, to:
acquire a scanning image of a target object during a positioning stage, and a respiratory signal of the target object; and
determine a tumor motion model based on a planning image of the target object, a position of a tumor of the target object in the scanning image, and the respiratory signal; wherein the tumor motion model is used to characterize an orientation change situation of the tumor of the target object during a respiratory process of the target object.

16. The electronic device according to claim 15, wherein the processor is further configured to:
in response to that a distance between a position of the tumor of the target object in the planning image and an imaging point is shorter than or equal to a preset threshold, determine the tumor motion model based on the planning image, the position of the tumor of the target object in the scanning image, and the respiratory signal.

17. The electronic device according to claim 15, wherein the scanning image comprises a plurality of projection images;
before determining the tumor motion model based on the planning image of the target object, the position of the tumor of the target object in the scanning image, and the respiratory signal, the processor is further configured to:
determine a reference projection image from the scanning image; wherein the reference projection image refers to an image containing the tumor of the target object among the plurality of projection images;
determine a target respiratory signal corresponding to a moment of harvesting the reference projection image from the respiratory signal; and
determine the tumor motion model based on the planning image, a position of the tumor of the target object in the reference projection image, and the target respiratory signal.

18. The electronic device according to claim 17, wherein the processor is further configured to:
acquire the reference projection image from projection images at a reference projection angle included in the scanning image according to a preset sampling interval.

19. The electronic device according to claim 18, wherein the reference projection angle is determined according to at least one of a first projection angle, a second projection angle, and a third projection angle;
wherein the first projection angle refers to a projection angular range where a path of a radiation beam emitted from a radiation source to the tumor of the target object does not contain a preset tissue;
the second projection angle refers to a projection angular range corresponding to a respiratory cycle during which a fitting degree between a motion trajectory of the tumor and a preset trajectory is highest;
the third projection angle refers to an available angular range for half field imaging.

20. The electronic device according to claim 19, wherein in a half field imaging mode, the reference projection angle is an intersection of the first projection angle and the third projection angle.
